(19) United States
(12) Patent Application Publication
(10) Pub. No.: US 2026/0096989 A1
(43) Pub. Date: Apr. 9, 2026

(54) LARGE-SCALE MANUFACTURING METHODS FOR AMINOGLYCOSIDES

(71) Applicant: Insmed Incorporated, Bridgewater, NJ (US)

(72) Inventor: Robert WORSHAM, Bridgewater, NJ (US)

(73) Assignee: Insmed Incorporated, Bridgewater, NJ (US)

(21) Appl. No.: 19/413,999

(22) Filed: Dec. 9, 2025

Related U.S. Application Data
(63) Continuation of application No. 18/825,766, filed on Sep. 5, 2024, now Pat. No. 12,521,345.
Application No. 18/825,766 is a continuation of application No. 17/052,070, now abandoned.
(60) Provisional application No. 62/665,564, filed on May 2, 2018.

(51) Int. Cl. A61K 9/1277; A61K 31/7036

(52) U.S. Cl. CPC A61K 9/1277; A61K 31/7036

(57) ABSTRACT

Provided herein is a method for the large-scale manufacture of liposomal drug formulations containing an aminoglycoside such as amikacin having advantageous lipid/drag characteristics. The method utilizes a particular relative flow rate ratio of lipid to drug streams to obtain liposomes with a high aminoglycoside encapsulation efficiency. The resulting liposomal drug formulations advantageously comprise an overall lipid-to-drug weight ratio of less than 1:1.

### CROSS REFERENCE TO RELATED APPLICATION

This application is a continuation of U.S. application Ser. No. 18/825,766, filed Sep. 5, 2024, which is a continuation of U.S. application Ser. No. 17/052,070, filed Oct. 30, 2020, which is a U.S. National Phase application of PCT/US2019/030404, filed May 2, 2019, which claims priority from U.S. Provisional Application Ser. No. 62/665,564, filed May 2, 2018, the disclosure of each of which is incorporated by reference herein in its entirety.

### BACKGROUND OF THE INVENTION

Liposomal drug formulations enable the ability to target and enhance the uptake of active agents at specific sites of disease. Such formulations have been developed to treat various pulmonary disorders, including those caused by pulmonary infections, where their characteristics make them an ideal choice for the inhalation delivery of anti-infective agents.

One such anti-infective agent, amikacin, has been packaged in liposomes, and has been studied in multiple clinical trials in adult patients for the treatment of refractory nontuberculous mycobacterial (NTM) lung disease cause by Mycobacterium avium complex (MAC). In a recent Phase 3 study of the amikacin liposome inhalation suspension (ALIS), it was demonstrated that the addition of ALIS to guideline based therapy (GBT) eliminated evidence of NTM lung disease caused by MAC in sputum by month 6 in 29% of patients, compared to 9% of patients on GBT alone.

Although liposomes containing a relatively high amikacin to lipid ratio have been prepared at the bench scale, it is well-known that it is not a routine matter to scale up such processes to produce, at the commercial manufacturing scale, liposomal formulations where parameters such as drug concentration, amount of lipid in the formulation, lipid-to-drug ratio, captured volume, drug leakage, viscosity, and particle size are consistently maintained within specification for clinical and/or commercial use.

The present invention addresses the need for a repeatable large-scale process for preparing liposomes containing an aminoglycoside antibiotic such as amikacin, and having a high aminoglycoside-to-lipid weight ratio (and in turn, a low lipid-to-aminoglycoside weight ratio) and superior encapsulation efficiency.

### SUMMARY OF THE INVENTION

In one aspect, the present invention provides a method for the large-scale manufacture of a liposomal aminoglycoside formulation comprising a lipid and an aminoglycoside (e.g., amikacin), with a high aminoglycoside loading relative to the lipid concentration (i.e., a high relative weight ratio of aminoglycoside-to-lipid). In particular, the lipid-to-aminoglycoside (e.g., amikacin) weight ratio (also referred to as the “L/D weight ratio”) in the liposomal suspension prepared according to method of the present invention is less than 1:1 upon completion of the process, for example between about 0.5:1 and about 0.9:1. In one embodiment, the lipid-to-aminoglycoside weight ratio of the liposomal suspension prepared according to the method of the present invention is about 0.7:1 (lipid:aminoglycoside) upon completion of the manufacturing process.

In another aspect, the present invention relates to a method for the large-scale manufacture of a liposomal drug formulation comprising a lipid and aminoglycoside (e.g., amikacin), wherein the aminoglycoside is contained within the liposome with a high encapsulation efficiency (e.g., an encapsulation efficiency of at least about 40% prior to washing to remove free aminoglycoside from the formulation).

In one embodiment, the method comprises mixing a first stream comprising a lipid with a second stream comprising an aminoglycoside to form a combined lipid-aminoglycoside stream. The combined lipid-aminoglycoside stream comprises liposomally encapsulated aminoglycoside, which in one embodiment, is formed at the intersection of the lipid stream and the aminoglycoside stream. In a further embodiment, the lipid-aminoglycoside stream is mixed with an aqueous saline solution in a reaction vessel (see, e.g., FIG. 1). In one embodiment, the aminoglycoside is present in an aqueous solution prior to the mixing step. In another embodiment, the lipid is present in an alcoholic solution, e.g., an ethanolic solution, prior to the mixing step. In a further embodiment, the lipid comprises a phospholipid and cholesterol. In one embodiment, the relative flow rate ratio of the second stream comprising aminoglycoside to the first stream comprising a lipid is about 1.5:1 (aminoglycoside stream:lipid stream) to about 2:1 (aminoglycoside stream:lipid stream). In a further embodiment, the lipid comprises dipalmitoylphosphatidylcholine (DPPC) and cholesterol.

In one embodiment, the aqueous saline solution is added to the reaction vessel via a third stream. In a further embodiment, the third stream is added to the reaction vessel at the same time as the combined lipid-aminoglycoside stream. In another embodiment, the third stream is added to the reaction vessel prior to the addition of the combined lipid-aminoglycoside stream to the reaction vessel. In another embodiment, the aqueous saline solution is at about room temperature prior to entering the reaction vessel. In one embodiment, the aqueous saline solution is about 1.5% aqueous sodium chloride.

In one particular aspect, the present invention provides a method for the large-scale manufacture of a liposomal drug formulation comprising a lipid and aminoglycoside (e.g., amikacin), wherein the aminoglycoside is encapsulated within or complexed with the liposome, prior to a washing step, at an encapsulation efficiency of at least 40%. Following the washing step, which in one embodiment, is carried out via tangential flow filtration, the weight ratio of lipid-to-aminoglycoside in the liposomal aminoglycoside formulation is less than 1:1, for example between about 0.5:1 and about 0.9:1 (e.g., about 0.7:1). In one embodiment of this method, the aminoglycoside is amikacin. In a further embodiment, the amikacin is present as amikacin sulfate.

In one embodiment, a first stream comprising a lipid is mixed with a second stream comprising an aminoglycoside to form a combined lipid-aminoglycoside stream (e.g., a lipid-amikacin stream) comprising liposomal aminoglycoside. In one embodiment, the liposomal aminoglycoside formulation is formed at the intersection of the two streams, i.e., upon formation of the combined lipid-aminoglycoside stream. In a further embodiment, the flow rate of the first stream comprising a lipid is from about 0.5 kg/min to about 1.5 kg/min and the flow rate of the second stream comprising aminoglycoside is from about 1 kg/min to about 2 kg/min. In a further embodiment, the flow rate of the first stream comprising a lipid is from about 3 kg/min to about 4 kg/min and the flow rate of the second stream comprising the aminoglycoside is from about 5 kg/min to about 7 kg/min. In another embodiment, the relative flow rate ratio of the second stream comprising aminoglycoside to the first stream comprising a lipid is about 1.5:1 (aminoglycoside stream flow rate:lipid stream flow rate) to about 2:1 (aminoglycoside stream flow rate:lipid stream flow rate). In yet another embodiment, the lipid comprises dipalmitoylphosphatidylcholine (DPPC) and cholesterol.

In one embodiment, the method for the large-scale manufacture of a liposomal drug formulation comprises mixing a first stream comprising a lipid with a second stream comprising aminoglycoside to form a combined lipid-aminoglycoside stream, and adding the combined lipid-aminoglycoside stream to a vessel comprising an aqueous saline solution. The aqueous saline solution, in one embodiment, is added to the reaction vessel via a third stream (see, e.g., FIG. 1).

In a further embodiment, when the flow rate of the first stream comprising a lipid is from about 0.5 kg/min to about 1.5 kg/min and the flow rate of the second stream comprising aminoglycoside is from about 1 kg/min to about 2 kg/min, the flow rate of the third stream is from about 0.5 L/min and about 2.0 L/min, for example, from about 1.0 L/min to about 2.0 L/min, e.g. from about 1.0 L/min to about 1.5 L/min, including about 1.25 L/min. In another embodiment, when the flow rate of the first stream comprising a lipid is from about 3 kg/min to about 4 kg/min and the flow rate of the second stream comprising aminoglycoside is from about 5 kg/min to about 7 kg/min, the flow rate of the third stream is from about 3 L/min and about 6 L/min, for example, from about 4 L/min to about 6 L/min, e.g. from about 4.5 L/min to about 5.5 L/min, including about 5 L/min.

As used herein, except where specifically stated otherwise, the term “aminoglycoside” is intended to include the aminoglycoside free base and any pharmaceutically acceptable salt thereof. For example, the term “amikacin” is intended to include amikacin free base and any pharmaceutically acceptable salt thereof (e.g., amikacin sulfate).

In one embodiment, the method for the large-scale manufacture of a liposomal aminoglycoside (e.g. amikacin) formulation comprises mixing a first stream comprising a lipid comprising a phospholipid with a second stream comprising aminoglycoside (e.g. amikacin) to form a combined lipid-aminoglycoside stream. In a further embodiment, the lipid-aminoglycoside stream is mixed with an aqueous saline solution in a reaction vessel. In one embodiment, the phospholipid is a phosphatidylcholine. In a further embodiment, the phosphatidylcholine is DPPC. In another embodiment, the lipid comprises a phospholipid and a sterol. In a further embodiment, the sterol is cholesterol. In one embodiment, the lipid comprises DPPC and cholesterol.

In one embodiment, the method for the large-scale manufacture of a liposomal aminoglycoside formulation comprises mixing a first stream comprising a lipid with a second stream comprising aminoglycoside, wherein the first stream is mixed with the second stream to form a combined lipid-aminoglycoside stream. The combined lipid-aminoglycoside stream comprises liposomal aminoglycoside. The liposomal aminoglycoside in one embodiment, is formed upon mixing the first stream and second stream, e.g., at the intersection of the two streams. In a further embodiment, the combined lipid-aminoglycoside stream is added to a reaction vessel and mixed with an aqueous saline solution. In a further embodiment, the aminoglycoside stream and the lipid stream are each maintained at a temperature from about 30° C. to about 50° C. prior to mixing. In a further embodiment, the aminoglycoside and lipid streams are each maintained at a temperature of from about 35° C. to about 45° C., for example from about 38° C. to about 42° C. prior to mixing. In one embodiment, the combined lipid-aminoglycoside stream is cooled upon entering the reaction vessel. In another embodiment, the combined lipid-aminoglycoside stream is cooled by the aqueous saline solution in the reaction vessel. In one embodiment, the reaction vessel is maintained at a temperature from about 25° C. and about 40° C., e.g., from about 27° C. to about 35° C. In another embodiment, the reaction vessel is maintained at a temperature of about 30° C. In another embodiment, the reaction vessel is maintained at a temperature of about 33° C.

In another aspect of the invention, a liposomal aminoglycoside formulation is manufactured on a large-scale according to a method provided herein. In one embodiment, the concentration of aminoglycoside (e.g. amikacin) present in the liposomal drug formulation so prepared is about 10 g/L or greater, for example from about 50 g/L to about 100 g/L, including about 60 g/L to about 80 g/L and about 65 g/L to about 75 g/L (e.g., about 20 g/L, about 30 g/L, 40 about g/L, about 50 g/L, about 60 g/L, about 70 g/L or about 80 g/L). In a further embodiment, the concentration of lipid present in the liposomal drug formulation so prepared is from about 10 g/L to about 100 g/L, including about 20 g/L to about 80 g/L and about 40 g/L to about 60 g/L (e.g. about 50 g/L). In another embodiment, the L/D ratio of a liposomal drug formulation manufactured on a large-scale according to a method provided herein is less than 1:1, for example between about 0.5:1 and about 0.8:1 (e.g. about 0.7:1).

In another embodiment, the liposomal drug formulation manufactured on a large-scale according to a method provided herein comprises liposome particles with a mean particle size (i.e. a mean diameter) of from about 200 nm to about 500 nm, for example from about 200 nm to about 400 nm (e.g. from about 250 nm to about 350 nm).

### BRIEF DESCRIPTION OF THE FIGURES

FIG. 1 depicting one embodiment of the invention for preparing a liposomal aminoglycoside formulation.

FIG. 2 shows the effect of relative lipid/amikacin flow rates on the resulting L/D ratio of various liposomal amikacin formulations.

### DETAILED DESCRIPTION OF THE INVENTION

In one aspect, the invention described herein relates to a method for manufacturing a liposomal aminoglycoside formulation on a large-scale. In one embodiment, the method comprises mixing a first stream comprising a lipid (also referred to herein as a “lipid stream”) with a second stream comprising an aminoglycoside such as amikacin (also referred to herein as a “drug stream”) to form a combined lipid-aminoglycoside stream, and the lipid-aminoglycoside stream is mixed with an aqueous saline solution in a reaction vessel. In some embodiments, the aqueous saline solution enters the reaction vessel via a third stream.

The mixing of the lipid and drug streams is effected such that a turbulent flow results when forming the combined lipid-aminoglycoside stream. A turbulent flow is conveniently achieved using an appropriate T-shaped or Y-shaped infusion module for “in-line” mixing of the lipid and drug streams.

The term “large-scale” means the use of at least about 5 kg aminoglycoside base starting material in the drug stream (calculated to at least about 5 kg aminoglycoside base if a pharmaceutically acceptable salt is used). In one embodiment, about 5 kg to about 50 kg aminoglycoside base starting material is used, for example about 5 kg to about 35 kg aminoglycoside base starting material. In one embodiment, at least about 8 kg aminoglycoside base starting material is used. In another embodiment, at least about 30 kg aminoglycoside base starting material is used. In one embodiment, the aminoglycoside is amikacin (e.g. amikacin sulfate).

The aminoglycoside used in the methods provided herein can be present as a pharmaceutically acceptable salt or as the free base. As provided above, in one embodiment, the aminoglycoside is amikacin, e.g., amikacin sulfate.

In another embodiment, the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin, or a combination thereof.

In yet another embodiment, the aminoglycoside is AC4437, amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycm, plazomicin, ribostamycin, sisomicm, rhodestreptomycin, sorbistin, spectinomycin, sporaricin, streptomycin, tobramycin, verdamicin, vertilmicin, or a combination thereof.

A “pharmaceutically acceptable salt” includes both acid and base addition salts. A pharmaceutically acceptable addition salt refers to those salts which retain the biological effectiveness and properties of the free bases, which are not biologically or otherwise undesirable, and which are formed with inorganic acids such as, but are not limited to, hydrochloric acid (HCl), hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid (e.g., as lactate), lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, acetic acid (e.g., as acetate), tartaric acid, thiocyanic acid, p-toluenesulfonic acid, trifluoroacetic acid (TFA), undecylenic acid, and the like. In one embodiment, the pharmaceutically acceptable salt is HCl, TFA, lactate or acetate. In one embodiment, the pharmaceutically acceptable salt is a sulfate salt, e.g., amikacin sulfate.

“Liposomal aminoglycoside formulation” refers to a lipid-aminoglycoside formulation wherein the lipid is in the form of a liposome and the aminoglycoside is encapsulated by the liposome bilayer, or complexed with the liposome bilayer. Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

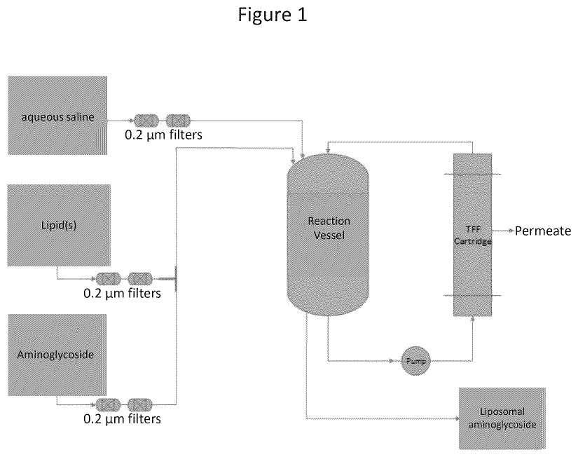

In one embodiment, the lipid-aminoglycoside formulation is manufactured via a method comprising a two-stream infusion process. In one embodiment, the method comprises mixing a first lipid stream with a second aminoglycoside stream in a T-shaped infusion module or Y-shaped infusion module. The terms “T-shaped infusion module,” and “Y-shaped infusion module” as used herein, refer to a T-shaped or Y-shaped chamber in which two or more streams are combined, for example, in which a lipid stream and a drug stream are combined to form a single lipid-aminoglycoside stream. See, e.g., the diagram at FIG. 1. It will be appreciated that the infusion module will have a bore size appropriate for the required rate of the lipid and drug streams used. Examples of suitable bore sizes include, but are not limited to, 3/16″ and ⅜″.

In one embodiment, the first stream (lipid stream) comprises an alcoholic (e.g., ethanolic) lipid solution. In one embodiment, the second stream (aminoglycoside stream) comprises an aqueous aminoglycoside solution (e.g., aqueous amikacin solution). In one embodiment, the first and second streams are mixed to form a combined lipid-aminoglycoside stream. In one embodiment, the first and second streams each enter the infusion module and the first and second streams are mixed in the infusion module. In a further embodiment, the combined lipid-aminoglycoside stream exits the infusion module and subsequently enters the reaction vessel. See FIG. 1.

In one embodiment, the combined lipid-aminoglycoside stream is mixed with an aqueous saline solution in a reaction vessel, e.g., the same reaction vessel that the combined lipid-aminoglycoside stream enters after exiting the infusion module. In one embodiment, the aqueous saline solution comprises about 0.5-2% aqueous sodium chloride solution (e.g., about 1.5%). In one embodiment, the saline solution is added to the reaction vessel prior to the combined lipid-aminoglycoside stream. In another embodiment, the saline solution is added to the reaction vessel at or about the same time as the combined lipid-aminoglycoside stream. In a further embodiment, the saline solution is added to the reaction vessel via a third stream. Thus, in some embodiments, the lipid-aminoglycoside formulation is manufactured via a method comprising a 3-stream infusion process. In some embodiments, the third stream is added to the reaction vessel separately from the combined lipid-aminoglycoside stream.

In one embodiment, the combined lipid-aminoglycoside stream comprises liposomal aminoglycoside, (e.g. amikacin), wherein the encapsulation efficiency of the aminoglycoside within the liposomes (or complexed to the liposomes) is at least about 40%. “Encapsulation efficiency”, as used herein, refers to the amount of aminoglycoside encapsulated or complexed with liposomes prior to a filtration step, e.g., tangential flow filtration of the liposomal aminoglycoside formulation to remove free aminoglycoside. For example, an encapsulation efficiency of between about 40% and about 70% (e.g., from about 45% to about 55%) can be achieved by mixing the lipid and aminoglycoside (e.g. amikacin) streams according to the method of this invention as herein described.

In one embodiment, the aminoglycoside stream and the lipid stream are each maintained at a temperature from about 30° C. to about 50° C. prior to mixing the two streams. In a further embodiment, the aminoglycoside and lipid streams are each maintained at a temperature of from about 35° C. to about 45° C., for example from about 38° C. to about 42° C. prior to mixing. In another embodiment, the combination of the lipid and aminoglycoside solutions exhibits exothermal behavior. The temperature of the combined lipid-aminoglycoside stream, in one embodiment, is from about 40° C. to 55° C. In a further embodiment, the temperature of the combined lipid-aminoglycoside stream is from about 45° C. to about 50° C. In another embodiment, the combined lipid-aminoglycoside stream is mixed with an aqueous saline solution in a reaction vessel, wherein the aqueous saline solution is maintained at room temperature prior to mixing with the combined lipid-aminoglycoside stream. In another embodiment, an aqueous saline solution is added to the reaction vessel via a third stream, wherein the third stream is maintained at room temperature prior to mixing with the combined lipid-aminoglycoside stream. In one embodiment, the combined lipid-aminoglycoside stream is cooled upon entering the reaction vessel. In another embodiment, the combined lipid-aminoglycoside stream is cooled by the aqueous saline solution in the reaction vessel. In another embodiment, the combined lipid-aminoglycoside stream is cooled upon entering the reaction vessel. In one embodiment, the reaction vessel is maintained at a temperature from about 25° C. and about 40° C., e.g., from about 27° C. to about 35° C. In another embodiment, the reaction vessel is maintained at a temperature of about 30° C. In another embodiment, the reaction vessel is maintained at a temperature of about 33° C.

In one embodiment, the lipid component of the liposomal drug formulation manufactured by the method provided herein comprises electrically net neutral lipids, positively charged lipids, negatively charged lipids, or a combination thereof. In another embodiment, the lipid component comprises electrically net neutral lipids. In a further embodiment, the lipid component consists essentially of electrically net neutral lipids. In even a further embodiment, the lipid is DPPC and cholesterol.

The lipids used in the manufacture of the liposomal formulations of the present invention can be synthetic, semi-synthetic or naturally-occurring lipids, including one or more of phospholipids, tocopherols, sterols, fatty acids, negatively-charged lipids and cationic lipids. In one embodiment, the lipid component consists of electrically neutral lipids, e.g., a sterol and a phospholipid.

In one embodiment, at least one phospholipid is present in the liposomal drug formulation. In one embodiment, the phospholipid is phosphatidylcholine (PC), phosphatidylglycerol (PG), phosphatidylinositol (PI), phosphatidylserine (PS), phosphatidylethanolamine (PE), phosphatidic acid (PA), soy phosphatidylcholine (SPC), soy phosphatidylglycerol (SPG), soy phosphatidylserine (SPS), soy phosphatidylinositol (SPI), soy phosphatidylethanolamine (SPE), and soy phosphatidic acid (SPA); hydrogenated egg and soya counterparts (e.g., hydrogenated egg phosphatidylcholine and hydrogenated soy phosphatidylcholine), phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The carbon chains on these fatty acids can be saturated or unsaturated, and the phospholipid may be made up of fatty acids of different chain lengths and different degrees of unsaturation.

In one embodiment, the lipid component of the liposomal drug formulation manufactured by the method provided herein comprises a phosphatidylcholine. For example, in one embodiment, the lipid component in the liposomal drug formulation comprises dipalmitoylphosphatidylcholine (DPPC). In one embodiment, the lipid component of the liposomal drug formulation comprises DPPC and a sterol, for example DPPC and cholesterol. Alternatively, the lipid consists essentially of DPPC and cholesterol, or consists of DPPC and cholesterol. In a further embodiment, the DPPC and cholesterol have a molar ratio in the range of from about 19:1 (DPPC:cholesterol) to about 1:1 (DPPC:cholesterol), or from about 9:1 (DPPC:cholesterol) to about 1:1 (DPPC:cholesterol), or from about 4:1 (DPPC:cholesterol) to about 1:1 (DPPC:cholesterol), or from about 2:1 (DPPC:cholesterol) to about 1:1 (DPPC:cholesterol). In even a further embodiment, the DPPC and cholesterol have a molar ratio of about 2:1 (DPPC:cholesterol).

Other examples of lipid components of the liposomal drug formulation manufactured by the method provided herein include, but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE).

Examples of sterol compounds in the liposomal drug formulation manufactured by the method provided herein include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols include tocopherols, esters of tocopherols including tocopherol hemi-succinates, salts of tocopherols including tocopherol hydrogen sulfates and tocopherol sulfates. The term “sterol compound” includes sterols, tocopherols and the like. Tocopherols and their water-soluble derivatives have been used to form liposomes, see, e.g., PCT Publication No. 87/02219.

In one embodiment, the concentration of lipid in the first stream is from about 10 g/L to about 50 g/L, or from about 10 g/L to about 30 g/L, or from about 15 g/L to about 25 g/L. In one embodiment, the concentration of lipid in the first stream is about 17 g/L, about 18 g/L, about 19 g/L, about 20 g/L, about 21 g/L, about 22 g/L, about 23 g/L, about 24 g/L or about 25 g/L. In one embodiment, the concentration of lipid in the first stream is about 20 g/L

In one embodiment, the concentration of aminoglycoside in the second stream (aminoglycoside stream) is from about 10 g/L to about 100 g/L; or from about 20 g/L to about 70 g/L; or from about 30 g/L to about 60 g/L; or from about 40 g/L to about 50 g/L. In one embodiment, the concentration of drug in the second stream is about 41 g/L, about 42 g/L, about 43 g/L, about 44 g/L, about 45 g/L, about 46 g/L, about 47 g/L, about 48 g/L, about 49 g/L or about 50 g/L. In one embodiment, the concentration of aminoglycoside in the second stream is about 45 g/L. In a further embodiment, the aminoglycoside is amikacin.

In one embodiment of the invention, the pH of the aminoglycoside stream is from 6 to about 7, or from about 6.5 to about 7.0. In a further embodiment, the pH of the aminoglycoside stream is about 6.7. The aminoglycoside stream pH may be adjusted to the appropriate pH using a suitable base, such as an alkali or alkaline earth metal hydroxide, e.g. sodium hydroxide.

In another embodiment, the aqueous saline solution comprises about 0.5% sodium chloride to about 3% sodium chloride, for example about 0.75%, about 1.0%, about 1.25%, about 1.5%, about 1.75%, about 2.0%, or about 2.5% sodium chloride. In one embodiment, the aqueous saline solution comprises about 1.5% sodium chloride.

In one embodiment, the flow rate of the lipid stream is from about 0.5 kg/min to about 1.5 kg/min and the flow rate of the aminoglycoside stream is from about 1 kg/min to about 2 kg/min. In a further embodiment, the flow rate of the lipid stream is from about 3 kg/min to about 4 kg/min and the flow rate of the drug stream is from about 5 kg/min to about 7 kg/min. In another embodiment, the relative flow rate ratio of the aminoglycoside stream to the lipid stream is about 1.5:1 (aminoglycoside stream flow rate:lipid stream flow rate) to about 2:1 (aminoglycoside stream flow rate:lipid stream flow rate).

In one embodiment, when the flow rate of the lipid stream is from about 0.5 kg/min to about 1.5 kg/min and the flow rate of the aminoglycoside stream is from about 1 kg/min to about 2 kg/min, the flow rate of the third stream comprising aqueous saline solution is from about 0.5 L/min and about 2.0 L/min, for example, from about 1.0 L/min to about 2.0 L/min, e.g., from about 1.0 L/min to about 1.5 L/min, including about 1.25 L/min. In another embodiment, when the flow rate of the lipid stream is from about 3 kg/min to about 4 kg/min and the flow rate of the aminoglycoside stream is from about 5 kg/min to about 7 kg/min, the flow rate of the third stream comprising the aqueous saline solution is from about 3 L/min and about 6 L/min, for example, from about 4 L/min to about 6 L/min, e.g. from about 4.5 L/min to about 5.5 L/min, including about 5 L/min.

In one embodiment of the invention, the lipid and aminoglycoside (e.g., amikacin) solutions are both filtered, for example through one or more (e.g., two in series) about 0.2 μm filters, prior to mixing into a combined stream (FIG. 1). Although FIG. 1 shows two filters in series, it should be noted that this number can be changed according to the preference of the user of the method. For example, one to five filters can be used to initially filter the lipid stream and the aminoglycoside stream.

In another embodiment, the aqueous saline solution (e.g., 1.5% saline solution) is also filtered, for example through one or more (e.g., two in series) about 0.2 μm filters, prior to mixing with the lipid-aminoglycoside combined stream in the reaction vessel. In a further embodiment, the liposomal suspension, comprising liposomes formed at the intersection of the lipid and aminoglycoside streams, and/or in the combined lipid-aminoglycoside stream, is concentrated within the reaction vessel using a recirculating filtration system such as diafiltration. As provided above, “encapsulation efficiency”, as used herein, refers to the amount of aminoglycoside encapsulated or complexed with liposomes prior to a filtration step, e.g., tangential flow filtration of the liposomal aminoglycoside formulation to remove free aminoglycoside. For example, an encapsulation efficiency of between about 40% and about 70% (e.g., from about 45% to about 55%) can be achieved by mixing the lipid and aminoglycoside (e.g. amikacin) streams according to the method of this invention as herein described.

In another embodiment, the resulting concentrated liposomal suspension is treated (i.e., “washed”) with additional aqueous saline solution (e.g., filtered 1.5% saline solution) and subjected to further filtration using a recirculating filtration system such as diafiltration until the liposomal suspension contains an appropriate final aminoglycoside concentration and substantially all of the free aminoglycoside is removed. In a further embodiment, three or more washes (e.g., 3, 4, 5 or 6 washes) are conducted to achieve the appropriate final aminoglycoside concentration.

In one embodiment, following washing, the concentration of aminoglycoside present in the liposomal aminoglycoside formulation manufactured on a large-scale according to a method provided herein is about 10 g/L or greater. In a further embodiment, aminoglycoside is present in the formulation at a concentration of about 20 g/L or greater. In a further embodiment, aminoglycoside is present in the formulation at a concentration of about 30 g/L or greater. In a further embodiment, aminoglycoside is present in the formulation at a concentration of about 40 g/L or greater. In a further embodiment, aminoglycoside is present in the formulation at a concentration of about 50 g/L or greater. In a further embodiment, aminoglycoside is present in the formulation at a concentration of about 60 g/L or greater. In a further embodiment, aminoglycoside is present in the formulation at a concentration of about 70 g/L or greater. In another embodiment, the aminoglycoside is present in the formulation at a concentration of from about 10 g/L to about 100 g/L. In a further embodiment, the aminoglycoside is amikacin. In one embodiment, the aminoglycoside is present in the formulation at a concentration of from about 50 g/L to about 100 g/L. In a further embodiment, the aminoglycoside is amikacin. In one embodiment, the aminoglycoside is present in the formulation at a concentration of from about 60 g/L to about 80 g/L. In a further embodiment, the aminoglycoside is amikacin. In yet another embodiment, the aminoglycoside is present in the formulation at a concentration from about 65 g/L to about 80 g/L. In a further embodiment, the aminoglycoside is amikacin. In yet another embodiment, the aminoglycoside is present in the formulation at a concentration from about 65 g/L to about 75 g/L. In a further embodiment, the aminoglycoside is amikacin. In another embodiment, amikacin is present in the formulation at a concentration of about 70 g/L. In a further embodiment, the aminoglycoside is amikacin.

In a further embodiment, following washing, the concentration of lipid present in the liposomal drug formulation manufactured on a large-scale according to a method provided herein is from about 10 g/L to about 100 g/L, including about 20 g/L to about 80 g/L and about 40 g/L to about 60 g/L (e.g., about 50 g/L).

In another embodiment, following diafiltration, the lipid-to-aminoglycoside weight ratio in a liposomal drug formulation manufactured on a large-scale according to a method provided herein is less than 1:1, for example between about 0.5:1 (lipid:aminoglycoside) and about 0.8:1 (lipid:aminoglycoside) (e.g., about 0.5:1 (lipid:aminoglycoside) or 0.6:1 (lipid:aminoglycoside) or 0.7:1 (lipid:aminoglycoside) or 0.8:1 (lipid:aminoglycoside)). In one embodiment, the lipid-to-aminoglycoside weight ratio is about 0.7:1 (lipid:aminoglycoside).

The liposomal aminoglycoside formulation manufactured on a large-scale according to a method provided herein comprises liposome particles with a mean particle size (i.e. a mean diameter) of from about 200 nm to about 500 nm, for example from about 200 nm to about 400 nm (e.g. from about 250 nm to about 350 nm). The liposome diameter may be measured using commercially available light scattering technology, for example by quasi-elastic light scattering using a Nicomp™ 380 submicron particle sizer (Nicomp, Santa Barbara, California USA).

The present invention is further illustrated by reference to the following Examples. However, it should be noted that these Examples, like the embodiments and aspects described above, are illustrative and are not to be construed as limiting the scope of the invention in any way.

### EXAMPLES

### Example 1: Manufacturing Process and Process Controls for Liposomal Amikacin

The manufacture of liposomal amikacin sulfate was conducted using an aseptic process that involves the preparation of three sterile solution streams, mixing the lipid and amikacin sulfate streams at appropriate flow rates via a T-connector infusion module, collecting the combined lipid-amikacin sulfate streams containing liposomes with encapsulated amikacin sulfate in a sterilized diafiltration (reaction) vessel, adding a stream of 1.5% aqueous sodium chloride at an appropriate flow rate to the diafiltration vessel, followed by diafiltration (including washing) and concentration of the resulting liposomal dispersion to form the final product.

a) Solution Preparation: Sufficient quantities of the following three solutions were prepared.
Amikacin sulfate solution: Amikacin sulfate in water for injection (WFI), pH adjusted with sodium hydroxide to 6.6-6.8.
Lipid solution: DPPC/cholesterol (2:1 w/w) in ethanol.
1.5% Sodium chloride solution: 1.5% Sodium chloride in WFI, pH adjusted to 6.6-6.8.

The solutions must be used within 24 hours of preparation.

b) Infusion/Initial Concentration: The amikacin sulfate solution and lipid solution were warmed and passed through separate sterilizing filters before flowing through an in-line T-connector infusion module at controlled rates of addition. The mixed streams were collected in a pre-sterilized reactor vessel. Simultaneously, 1.5% aqueous sodium chloride solution was passed through a sterilizing filter and introduced as a stream at an appropriate flow rate into the reactor vessel. At this stage, the solution may be sampled for the level of amikacin encapsulation.

c) Diafiltration: Diafiltration was conducted. This step functions to remove the ethanol from the bulk solution and to wash away any “unentrapped” or free amikacin sulfate.

d) Final Concentration: Using an in-process test result, the bulk solution was concentrated to an appropriate concentration level of amikacin sulfate. After concentration is complete, confirmatory tests for concentration of amikacin sulfate and L/D ratio may be performed.

Table 1 describes experiments (A) and (B), performed according to the general method of Example 1. In (A), a ⅜″ T-connector infusion module is used. In (B), a 3/16″ T-connector infusion module is used.

TABLE 1

| | | | | Amikacin | Amikacin | | | | Amikacin |
|---|---|---|---|---|---|---|---|---|---|
| [symbol] | Amikacin | | | sulfate | sulfate | | Lipid | | sulfate |
| [symbol] | calculated | | | solution | solution | Lipid solution | solution | L/D | concentration |
| [symbol] | free base | DPPC | Cholesterol | concentration | flow rate | concentration | flow rate | ratio | obtained |
| [symbol] | weight (kg) | weight (kg) | weight (kg) | (g/L) | (kg/min) | (g/L) | (kg/min) | obtained | (mg/mL) |
| [symbol] | 30.428 | 7.33 ± | 3.67 ± | 45 | 5.64 | 20 | 3.62 | 0.72 | 70 ± 3 |
| [symbol] | | 0.050 | 0.050 | | | | | | |
| [symbol] | 8.250 | 1.666 ± | 0.834 ± | 45 | 1.464 | 20 | 0.851 | 0.68 to | 70 ± 3 |
| [symbol] | | 0.001 | 0.001 | | | | | 0.74 | |

[symbol]

indicates data missing or illegible when filed

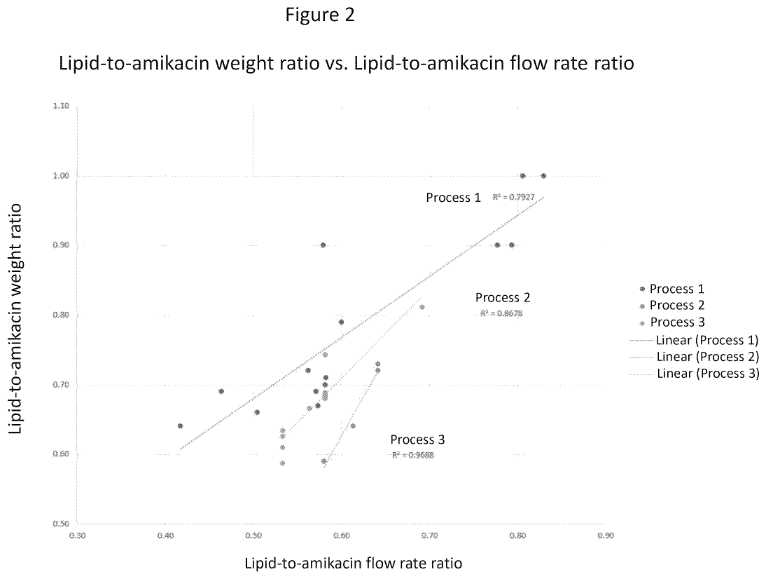

In additional experiments, generally following the process of Example 1, the lipid and amikacin stream (flow) rates were varied, and the resulting concentrations of lipid and amikacin in the liposomal formulations were measured. The L/D ratio for each experiment was calculated and the results presented in FIG. 2. The results provide guidance for an optimal relative lipid/amikacin flow rate to achieve a preferred L/D ratio.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A large-scale method of preparing a liposomal aminoglycoside formulation comprising a lipid and an aminoglycoside, wherein the overall lipid-to-drug weight ratio is less than 1:1, the method comprising:
(a) mixing a first stream comprising the lipid with a second stream comprising the aminoglycoside to form a combined lipid-aminoglycoside stream,
(b) mixing the lipid-aminoglycoside stream of Step (a) with an aqueous saline solution in a reaction vessel, and
(c) washing the product of Step (b) comprising the liposomal aminoglycoside formulation to remove unencapsulated aminoglycoside,
wherein the relative flow rate ratio of the second stream to the first stream is about 1.5:1 to about 2:1.

2. The method of claim 1, wherein the second stream comprises an aqueous solution of amikacin.

3. The method of claim 1, wherein the first stream comprises an alcoholic solution of lipid.

4. The method of claim 1, wherein the aqueous saline solution is added to the reaction vessel via a third stream.

5. The method of claim 4, wherein the third stream is added to the reaction vessel at the same time as the lipid-aminoglycoside stream.

6. The method of claim 4, wherein the third stream is added to the reaction vessel prior to the lipid-aminoglycoside stream.

7. The method of any one of claims 1-6, wherein the flow rate of the first stream is from about 0.5 kg/min to about 1.5 kg/min, and the flow rate of the second stream is from about 1 kg/min to about 2 kg/min.

8. The method of any one of claims 1-6, wherein the flow rate of the first stream is from about 3 kg/min to about 4 kg/min, and the flow rate of the second stream is from about 5 kg/min to about 7 kg/min.

9. The method of claim 6, wherein the flow rate of the first stream is from about 0.5 kg/min to about 1.5 kg/min, the flow rate of the second stream is from about 1 kg/min to about 2 kg/min, and the flow rate of the third stream is from about 1 L/min to about 2 L/min.

10. The method of claim 6, wherein the flow rate of the first stream is from about 3 kg/min to about 4 kg/min, the flow rate of the second stream is from about 5 kg/min to about 7 kg/min, and the flow rate of the third stream is from about 3 L/min to about 6 L/min.

11. The method of any one of claims 1-10, wherein the aqueous saline solution is 1.5% sodium chloride.

12. The method of any one of claims 1-11, wherein the aminoglycoside is a pharmaceutically acceptable salt of the aminoglycoside.

13. The method any one of claims 1-12, wherein the lipid comprises a phospholipid.

14. The method of claim 13, wherein the phospholipid is a phosphatidylcholine.

15. The method of claim 14, wherein the phosphatidylcholine is dipalmitoylphosphatidylcholine (DPPC).

16. The method of any one of claims 1-15, wherein the lipid comprises a sterol.

17. The method of claim 16, wherein the sterol is cholesterol.

18. The method of any one of claims 1-17, wherein the lipid comprises DPPC and cholesterol.

19. The method of any one of claims 1-18, wherein the drug and lipid streams are each maintained at a temperature from about 30° C. to about 50° C. prior to mixing.

20. The method of claim 19, wherein the drug and lipid streams are each maintained at a temperature from about 35° C. to about 45° C. prior to mixing.

21. The method of any one of claims 1-20, wherein the temperature of the combined lipid-aminoglycoside stream is cooled by the aqueous saline solution in the reaction vessel.

22. The method of any one of claims 1-21, wherein following Step (b) liposomes are prepared with an aminoglycoside encapsulation efficiency of at least 40%.

23. The method of any one of claims 1-22, wherein liposomes are formed in the combined lipid-aminoglycoside stream.

24. The method of any one of claims 1-23, for the preparation of a liposomal drug formulation where the overall lipid-to-drug weight ratio is about 0.7:1.

25. The method of any one of claims 1-23, wherein the washing Step (c) is performed using 1.5% aqueous sodium chloride solution.

26. The method of claim 25, wherein the washing Step (c) is repeated, and the product concentrated to provide a liposomal drug formulation with the aminoglycoside present at a concentration of from about 60 g/L to about 80 g/L.

27. The method of any one of claims 1-26, wherein the aminoglycoside is arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin, or a combination thereof.

28. The method of any one of claims 1-26, wherein the aminoglycoside is AC4437, amikacin, apramycin, arbekacin, astromicin, bekanamycin, boholmycin, brulamycin, capreomycin, dibekacin, dactimicin, etimicin, framycetin, gentamicin, H107, hygromycin, hygromycin B, inosamycin, K-4619, isepamicin, KA-5685, kanamycin, neomycin, netilmicin, paromomycm, plazomicin, ribostamycin, sisomicm, rhodestreptomycin, sorbistin, spectinomycin, sporaricin, streptomycin, tobramycin, verdamicin, vertilmicin, or a combination thereof

29. The method of any one of claims 1-26, wherein the aminoglycoside is amikacin.

30. The method of claim 29, wherein the amikacin is amikacin sulfate.

31. A liposomal drug formulation manufactured by the method of any one of claims 1-30.

32. The liposomal drug formulation of claim 31, wherein the aminoglycoside is present at a concentration from about 60 g/L to about 80 g/L.

33. The liposomal drug formulation of claim 31, wherein amikacin is present at a concentration of about 70 g/L.

34. The liposomal drug formulation of any one of claims 31-33, wherein the lipid is present at a concentration from about 40 g/L to about 60 g/L.

35. The liposomal drug formulation of claim 34, wherein the lipid is present at a concentration of about 50 g/L.
